(19) United States
(12) Patent Application Publication
(10) Pub. No.: US 2022/0362497 A1
(43) Pub. Date: Nov. 17, 2022

(54) MONITORING DEVICE AND SYSTEM

(71) Applicant: Ion Heat SAS, Rionegro (CO)

(72) Inventors: ANDRES BERNAL DUQUE, Rionegro (CO); JUAN DAVID RENDON VELASQUEZ, Rionegro (CO); JUAN DANIEL ISAZA BETANCOURT, Rionegro (CO)

(21) Appl. No.: 17/322,357

(22) Filed: May 17, 2021

(51) Int. Cl. A61M 16/00; A61M 16/08; A61M 16/10

(52) U.S. Cl. CPC A61M 16/024; A61M 16/0833; A61M 16/1005; A61M 16/0003; A61M 2230/42; A61M 2016/1025; A61M 2205/502; A61M 2016/003; A61M 2016/0027

(57) ABSTRACT

The present disclosure relates to a monitoring device for measuring and monitoring breathing parameters and, optionally, oxygenation and/or vital sign parameters of a mechanically ventilated patient, the monitoring device being removably arrangeable at a portion of a ventilator breathing circuit provided between and in fluid connection with a mechanical ventilator and an airway of the patient. The monitoring device comprises a first sensor arrangeable at the fluid connection for measuring parameters related to an airflow in the fluid connection to obtain measurement data; a processor adapted to receive the measurement data from the first sensor and configured to process the measurement data into at least one breathing parameter; and a transmitter adapted to transmit data comprising the at least one breathing parameter to an external device.

### FIELD OF THE DISCLOSURE

The present disclosure relates to a monitoring device and system for measuring and monitoring breathing parameters of a mechanically ventilated patient, and to a method for measuring and remote monitoring of breathing parameters of a mechanically ventilated patient.

### BACKGROUND OF THE DISCLOSURE

The covid-19 pandemic created a shortage of mechanical ventilators to be used in intensive care units (ICUs) around the world as the number of patients that needed mechanical ventilation increased significantly.

Mechanical ventilators have been in use for a long time and have been under constant development over the years. Particularly, in view of the Covid-19 pandemic, ventilator manufacturers accelerated to increase their manufacturing capabilities and new initiatives arose around the world to develop and manufacture such equipment. In parallel to the new initiatives, and in order to meet the demand for mechanical ventilators at the ICUs, inventories of any ventilator equipment usable was made, resulting in a wide variety of mechanical ventilators, both in terms of age and provider, being used at care centers.

However, while the number of ventilators provided at ICUs increased, the demand for on-site intensivists or clinicians that can operate them correctly also increased to such an extent that the number available could not meet the demand.

Tele-ICU is a growing concept around the world, where an off-site intensivist interacts with bedside staff to consult about procedures necessary for patient care. So, when an intensivist can monitor many ICU units sharing information electronically, and consults the bedside staff remotely, tele-ICU helps solving the shortage of such professionals.

Vital signs patient monitors often include the capability of remote monitoring, giving the institution the possibility of having a centralized control room for a professional to monitor many ICU beds. Mechanical ventilators, on the other hand, do not have such a level of connectivity and just recently they have started to incorporate such technology in new models.

Individual manufacturers have started to include the possibility of remote monitoring in their own equipment. A problem remains, however, since only newer mechanical ventilators from particular providers offer this possibility. So, for any institution, implementing remote monitoring of their mechanical ventilators creates a problem as large as the number of different models and brands of ventilators that are available in the institution.

### SUMMARY OF THE DISCLOSURE

It is an object to mitigate, alleviate, or eliminate one or more of the above-identified deficiencies in the art and disadvantages singly or in any combination, and to solve at least the above-mentioned problem.

To better address this concern, according to a first aspect of the disclosure there is provided a monitoring device for measuring and monitoring breathing parameters and, optionally, oxygenation and/or vital sign parameters of a mechanically ventilated patient. The monitoring device is removably arrangeable at a portion of a ventilator breathing circuit provided between and in fluid connection with a mechanical ventilator and an airway of the patient. The monitoring device comprises a first sensor arrangeable at the fluid connection for measuring at least one parameter related to an airflow in the fluid connection to obtain measurement data; a processor adapted to receive the measurement data from the first sensor and configured to process the measurement data into at least one breathing parameter; and a transmitter adapted to transmit data comprising the at least one breathing parameter to an external device.

The monitoring device may be advantageous as it allows obtaining and transmitting breathing parameters of a patient being mechanically ventilated to an external device, independently of the type of mechanical ventilator used by the patient. The monitoring device being removably arrangeable at a portion of a ventilator breathing circuit between and in fluid connection with a mechanical ventilator and an airway of the patient is thus advantageous since it can be used together with any mechanical ventilator. The ventilator breathing circuit provided between the mechanical ventilator and the airway of the patient is also generally called the ventilation patient circuit, and may further be referred to herein as the patient circuit. The first sensor being arrangeable in the fluid connection, e.g., connected in-line with the patient circuit, measures and transmits measurement data related to the air flow and airway pressure during mechanical ventilation to the processor. The processor processes the data into at least one breathing parameter which is then transmitted by the transmitter to an external device. This provides a compact device which is easily handled and installed at any mechanical ventilator. The first sensor is generally connected to the processor by a tube or a wire. However, providing a first sensor which transmits the measurement data wirelessly to the processor is also conceivable within the concept of the present disclosure. Transmission of the data comprising breathing parameters to the external device may be wireless or over a wired connection.

According to some embodiments, the first sensor is a flow sensor. Arranging the flow sensor in the fluid connection thus allows measuring parameters related to the air flow and pressure there through.

According to some embodiments, the flow sensor is a differential pressure flow sensor. The differential pressure flow sensor is arranged in the fluid connection between the mechanical ventilator and the patient such that a gas flow passes through the sensor, creating a differential pressure between two measuring ports included in the sensor. Based on the measurements provided by the differential pressure flow sensor in the fluid connection, several breathing parameters of the patient being mechanically ventilated can be computed by the processor. According to some embodiments, the at least one breathing parameter comprises ventilatory mechanics data. The ventilatory mechanics data may comprise at least one of airway pressure, gas flow, respiratory rate, inhale to exhale ratio, inspiratory time, expiratory time, tidal volume, peak inspiratory pressure (PIP), and positive end expiratory pressure (PEEP). The ventilatory mechanics data may comprise several or all of the aforementioned data. According to this embodiment, the differential pressure flow sensor is generally connected to the processor by a tube. More particularly, the differential pressure flow sensor can be connected to the processor by two tubes, thereby providing measurement data to the processor from two different points in the sensor, which allows obtaining measurements of the differential pressure. However, providing a differential pressure flow sensor which transmits the measurement data wirelessly to the processor is also conceivable within the concept of the present disclosure

According to some embodiments, the monitoring device further comprises a second sensor arrangeable at the fluid connection for measuring at least one parameter related to an airflow in the fluid connection to obtain second measurement data, wherein the processor is adapted to receive the second measurement data from the second sensor and is configured to process the second measurement data into at least one breathing parameter.

According to some embodiments, the second sensor is a capnography sensor. This allows obtaining a measurement of the partial pressure of carbon dioxide (CO₂) in the respiratory gas, which may be advantageous. A non-limiting example of a capnography sensor is an inline infrared sensor. Another non-limiting example is a gas sampling sensor which uses an internal capnography module.

According to some embodiments, the second sensor is an oxygen sensor. This allows obtaining a measurement of the oxygen concentration in the respiratory gas, and more particularly of the fraction of inspired oxygen (FiO₂), which may be advantageous.

According to an embodiment comprising a second sensor which is a capnography sensor, the monitoring device further comprises a third sensor arrangeable at the ventilator breathing circuit for measuring at least one parameter related to an airflow therein to obtain third measurement data, and wherein the processor is adapted to receive the third measurement data from the third sensor and configured to process the third measurement data into at least one breathing parameter. In a preferred embodiment, the third sensor is an oxygen sensor. The oxygen sensor may be arranged at the inspiratory limb of the ventilator breathing circuit, and thereby allows measuring the oxygen concentration of the, by the patient, inspired gas. A breathing parameter obtainable thereby is, thus, the fraction of inspired oxygen, FiO₂. It is thereby possible, within the inventive concept, to provide both a capnography sensor and an oxygen sensor for obtaining measurements of the partial pressure of CO₂ and of the fraction of inspired oxygen (FiO₂), in addition to the parameters obtainable by the differential pressure flow sensor. Thus, any combination of the differential pressure flow sensor, the capnography sensor, and the oxygen sensor may be provided within the context of the present disclosure, wherein in any case, each of the sensors are connected to the processor of the monitoring device.

According to some embodiments, the monitoring device further comprises a pulse oximeter for measuring at least one parameter related to pulse and/or oxygenation of the patient to obtain fourth measurement data, wherein the processor is adapted to receive the fourth measurement data from the pulse oximeter and process the fourth measurement data into at least one oxygenation parameter, and wherein the transmitter is adapted to transmit data comprising the at least one oxygenation parameter.

Within the context of the present disclosure, parameters obtainable by the pulse oximeter will be referred to as oxygenation parameters, and includes at least blood oxygen levels via an oxygen saturation measurement called peripheral capillary oxygen saturation (SpO₂) and heart rate. Other vital signs may also be obtainable with the pulse oximeter, or with another vital sign sensor that is capable of measuring vital signs of a patient in a non-invasive manner, such as a thermometer and/or a sphygmomanometer, and is connectable to the processor such that the processor receives measurement data therefrom for processing. The monitoring device may comprise a vital sign sensor for measuring, in a non-invasive manner, at least one parameter related to a vital sign of the patient to obtain measurement data, wherein the processor is adapted to receive the measurement data from the vital sign sensor and process the measurement data into at least one vital sign parameter, and the transmitter is adapted to transmit data comprising the at least one vital sign parameter.

According to some embodiments, the portion of the ventilator breathing circuit at which the measuring device is removably arrangeable is a y-piece of a patient circuit of a mechanical ventilator. More particularly, the measuring device is removably arrangeable at the portion of the patient circuit by the first sensor being removably arrangeable thereat. The first sensor is preferably arranged at the patient end of the y-piece of the ventilator breathing circuit provided between the mechanical ventilator and the patient. Optionally, the monitoring device comprises at least one second sensor, e.g. a capnography sensor and/or an oxygen sensor, which is/are also removably arrangeable at a portion of the ventilator breathing circuit between the ventilator and the patient. For example, the second sensor(s) can be arranged at the y-piece of the ventilator breathing circuit. Alternatively, according to an embodiment of the monitoring device comprising an oxygen sensor, the oxygen sensor is removably arranged at and in fluid connection with the inspiratory limb of the ventilator breathing circuit. This allows measuring the oxygen concentration of the inspiratory gas, e.g., the fraction of inspired oxygen. Arranging the first sensor and optionally any one of a capnography sensor and an oxygen sensor at a different portion of the ventilator breathing circuit provided between the mechanical ventilator and the patient, e.g., the patient circuit, is also conceivable within the inventive concept. For example, a separate adaptor may be provided, to which at least the first sensor is removably arrangeable, the separate adaptor being arrangeable inline and, thus, in fluid connection with the patient circuit. Preferably, the first sensor and optionally a second sensor and/or a third sensor is/are arranged on the patient side of the ventilator breathing circuit.

Further, according to some embodiments, any one of the first sensor, the second sensor, and the pulse oximeter is releasably connected to the processor. According to some embodiments, the processor and the transmitter may be arranged in a housing to which any one of the first sensor, the second sensor, and the pulse oximeter is connectable. The housing may also be referred to as a monitoring unit, since it is where the measurement data obtained from the at least first sensor is processed into breathing parameters, which are then transmitted for remote monitoring. This provides a compact monitoring device which is easy to handle and install, as the first sensor and optionally the second sensor and/or the pulse oximeter only needs to be connected to the processor through a corresponding port in the housing, and then arranged at a portion of the ventilator breathing circuit provided between the mechanical ventilator and the airway of the patient and optionally, for the pulse oximeter, at the body of the patient, e.g. the finger or the ear, in order for the monitoring device to be operable for monitoring breathing parameters and optionally oxygenation parameters of the patient. Thus, a monitoring device which is external to the mechanical ventilator and which can be installed at any patient circuit, independent of the type of mechanical ventilator used, is provided. This is further advantageous in that the first sensor, and optionally any of the second sensor and/or the pulse oximeter is easily exchangeable and may thus be replaced by a new sensor when necessary. The new sensor is then simply arranged at the desired portion of the ventilator breathing circuit and/or the body of the patient and connected to the processor through the corresponding port of the housing, and the monitoring device is operable.

According to some embodiments, the monitoring device comprises an internal sensor arranged in a housing of the monitoring device and adapted to receive a gas sample from the ventilator breathing circuit for measuring at least one parameter related to the gas sample to obtain measurement data, and wherein the processor is adapted to receive the measurement data from the internal sensor and configured to process the measurement data into at least one breathing parameter.

The internal sensor is according to an embodiment an internal capnography sensor. In this embodiment, a gas sample is provided to the internal capnography sensor in the housing through a fluid connection of the internal capnography sensor with the patient circuit. For example, the fluid connection can be embodied by a tube arranged to extend between a port of the housing with which the internal capnography sensor is fluidly connected and a portion of the patient circuit, such to provide a fluid connection there between. The fluid connection may be provided with valves for controlling e.g. the sampling of gas.

According to some embodiments, the internal sensor is an internal oxygen sensor. In this embodiment, a gas sample is provided to the internal oxygen sensor in the housing through a fluid connection of the internal oxygen sensor with the patient circuit. For example, the fluid connection can be embodied by a tube arranged to extend between a port of the housing with which the internal oxygen sensor is fluidly connected and a portion of the patient circuit, such to provide a fluid connection there between. The fluid connection may be provided with valves for controlling e.g. the sampling of gas.

According to some embodiments, the monitoring device comprises an internal capnography sensor and an internal oxygen sensor, and the internal capnography sensor is adapted to receive a gas sample from the patient circuit. The gas sample from the patient circuit may be obtained by arranging an external capnography gas sample sensor at the patient circuit and connecting it to a port of the housing with which the internal capnography sensor is in fluid connection. According to this embodiment, the internal oxygen sensor is configured to receive a gas sample from the internal capnography sensor to measure the oxygen concentration therein. More particularly, the monitoring device further comprises directional valves and the internal oxygen sensor is configured to receive a gas sample from the internal capnography sensor, which may be an internal gas sampling capnography module, through directional valves which are arranged to allow flow through the internal oxygen sensor when there is evidence that the gas sample corresponds to a gas delivered in the inspiration phase of the mechanical ventilation of the patient. Such evidence may for example correspond to a CO₂ concentration, measured by the internal capnography sensor, at the lowest level, thus indicating that the sample corresponds to fresh gas. The processor is in this embodiment adapted to receive measurement data thereby obtained by the oxygen sensor for processing. This provides an efficient monitoring device capable of obtaining and transmitting several breathing parameters. The monitoring device, according to this embodiment, is further easily installed as only the first sensor needs to be arranged at a portion of the patient circuit and a fluid connection needs to be established between the patient circuit and the internal capnography sensor. This latter fluid connection may for example be established by means of a hose connected at one end to a port of the housing, which is fluidly connected with the internal capnography sensor, and at the other end to the ventilator breathing circuit provided between the mechanical ventilator and the patient.

According to some embodiments, the device comprises a second sensor removably arrangeable at a portion of the patient circuit, and an internal sensor adapted to receive a gas sample from the patient circuit. The internal sensor of this embodiment may for example be an internal oxygen sensor.

According to some embodiments comprising an internal oxygen sensor, the monitoring device is removably connected to a T-piece provided at the inspiratory limb of the ventilator breathing circuit via a tubing. The internal oxygen sensor is here connected to the tubing by a port, e.g. a pneumatic port, in the housing of the monitoring device, such that a gas sample flows from the inspiratory limb of the ventilator breathing circuit to the oxygen sensor, which measures the oxygen concentration in the gas. This allows measuring the fraction of inspired oxygen. The processor is in this embodiment adapted to receive measurement data from the oxygen sensor for processing.

According to some embodiments, the processor comprises an encryptor (which may alternatively be called an encryption unit) for encrypting the breathing parameters and/or the oxygenation parameters to encrypted data, wherein the transmitter is adapted to transmit the encrypted data to the external device. This provides secure transmission of the data to the external device, which is advantageous. The external device may thus comprise a decryptor for decrypting the received data.

The transmission of the data, encrypted or not, is according to some embodiments wireless. According to some embodiments, the transmission of the data from the transmitter to the external device is over a wired connection.

According to a second aspect, there is provided a system for measuring and remote monitoring of breathing parameters and, optionally, oxygenation parameters and vital signs of a mechanically ventilated patient, the system comprising a monitoring device as disclosed herein associated with the patient, an external device configured to receive data from the monitoring device, the external device comprising a network server, and an intermediary device connected to the network server and comprising a user interface for displaying the breathing parameters and patient information, and, optionally, oxygenation parameters.

The system may be advantageous as it allows remote monitoring of a mechanically ventilated patient independently of the mechanical ventilator the patient is connected to.

According to some embodiments, the system further comprises an external storage device adapted to receive and store data transmitted from the transmitter of the monitoring device. This is advantageous as it provides a safe storage of the parameters processed and transmitted by the monitoring device.

According to some embodiments, the system comprises a plurality of monitoring devices, each associated with a respective patient, and the external device is configured to receive data from the plurality of monitoring devices. This allows transmitting breathing parameters, and optionally vital parameters, from several patients to the external device simultaneously, such that several patients may be remotely monitored at the same time by using one sole external device.

According to some embodiments, the user interface is configured to display breathing parameters for a plurality of patients. This provides an effective remote monitoring of breathing parameters of several patients simultaneously, such that a single off-site intensivist may monitor several patients which are being treated at different sites at the same time. Thus, specialist expertise is made more accessible.

According to some embodiments, the system comprises a plurality of intermediary devices connected to the network server. This is advantageous as it allows displaying breathing parameters of different patients at user interfaces of different intermediary devices.

According to a third aspect, there is provided a method for measuring and remote monitoring of breathing parameters and, optionally, oxygenation parameters, of a mechanically ventilated patient. The method comprises the steps of removably arranging a first sensor at a portion of a ventilator breathing circuit provided between and in fluid connection with a mechanical ventilator and the airway of the patient; obtaining measurement data, via the first sensor, related to an airflow and airway pressure in the fluid connection; receiving, by a processor, the measurement data from the first sensor; processing, by the processor, the measurement data into at least one first breathing parameter; and transmitting, by a transmitter connected to the processor, data comprising the at least one first breathing parameter to an external device.

The method may be advantageous as it allows for measuring and remote monitoring of breathing parameters and/or oxygenation parameters of a patient being mechanically ventilated independent of the mechanical ventilator used by the patient. The data, or the set of data, obtained by the first sensor, which is removably arranged in-line with a ventilator breathing circuit provided between the mechanical ventilator and the patient, is received and processes by a processor into at least one first breathing parameter. The breathing parameter(s) are then transmitted by the transmitter to an external device.

According to some embodiments, the method further comprises removably arranging a second sensor at the portion of the ventilator breathing circuit; obtaining second measurement data, via the second sensor, related to the airflow in the fluid connection; receiving, by the processor, the second measurement data from the second sensor; processing, by the processor, the measurement data into at least one second breathing parameter; and wherein the transmitting comprises transmitting data comprising the first and the second breathing parameters to an external device. This allows obtaining additional breathing parameters, provided through the second measurement data, or the second set of measurement data, for remote monitoring of the patient.

According to some embodiments, the method further comprises removably arranging a fourth sensor at a body of the patient; obtaining fourth measurement data, via the fourth sensor, related to a pulse and/or an oxygenation of the patient; receiving, by the processor, the fourth measurement data from the fourth sensor; processing, by the processor, the measurement data into at least one oxygenation parameter; and wherein the transmitting comprises transmitting data comprising the first and the second breathing parameters, and the at least one oxygenation parameter to an external device.

This is advantageous since it allows measuring and remote monitoring of vital parameters, in the form of e.g. oxygenation parameters obtained from a pulse oximeter, of the patient in addition to breathing parameters.

According to some embodiments, the method further comprises encrypting the at least one first breathing parameter, the at least one second breathing parameter, the at least one oxygenation or vital sign parameter, and/or the data before the transmitting to the external device. This may be advantageous to provide a safe transmission of the data to the external device.

According to some embodiments, the method further comprises decrypting, by the external device, data received from the transmitter.

According to some embodiments, the first sensor is a differential pressure flow sensor. This allows obtaining parameters from one sole measurement including one or more of airway pressure, gas flow, respiratory rate, inhale to exhale ratio, inspiratory time, expiratory time, tidal volume, peak inspiratory pressure, and positive end expiratory pressure.

According to some embodiments, the second sensor is one of a capnography sensor and an oxygen sensor. The capnography sensor provides measuring and monitoring of the concentration or partial pressure of carbon dioxide (CO₂) in the respiratory gas, which is advantageous. The oxygen sensor allows measuring and monitoring of the oxygen concentration in the inspiratory gas, which is advantageous.

According to some embodiments, the method further comprises removably arranging two different second sensors at respective portions of the ventilator breathing circuit provided between the mechanical ventilator and the patient, obtaining measurement data from the two different second sensors, and receiving, by the processor, the measurement data from the two different second sensors. The second sensors may for example be a capnography sensor and an oxygen sensor. In this embodiment, the transmitting thus comprises transmitting data originating from the first sensor, the capnography sensor, and the oxygen sensor, to an external device.

According to some embodiments, the fourth sensor is a pulse oximeter. The pulse oximeter allows measuring the pulse and oxygen saturation in the blood of the patient. The parameters measured by and obtained from the pulse oximeter are generally referred to herein as oxygenation parameters.

According to some embodiments, the method further comprises receiving, by an intermediary device connected to the external device, data related to a user interface for displaying the breathing parameters and optionally vital parameters; and displaying, by the intermediary device, the user interface. The breathing parameters transmitted in the form of e.g. airway pressure, gas flow, inspiratory and expiratory volume, respiratory rate, PEEP value, and inspiration and expiration times from the transmitter to the external device can thus be transmitted to the user interface and displayed in the form of e.g. graphs for the monitoring by an off-site intensivist. This allows for an intensivist to remotely monitor vital parameters of a patient and, by communicating with the bedside staff, provide necessary care instructions for the patient.

According to some embodiments, the step of decrypting data is carried out by the intermediary device. In a particular embodiment, the encrypted data transmitted to the external device is further encrypted, by the external device, to provide a double encryption of the data. This provides a safe handling of the data. When solicited by the intermediary device, the double encryption is decrypted by the external device, before transmittal to the intermediary device. Final decryption of the data is then made by the intermediary device, for displaying the at least one breathing parameter and optionally oxygenation or vital sign parameters on the user interface.

According to some embodiments, the user interface is configured to display vital parameters for a plurality of patients.

According to some embodiments, the obtaining comprises continuously obtaining measurement data and the transmitting comprises continuously transmitting data to the external unit for continuous monitoring of the vital parameters. This allows continuous real-time remote monitoring of the vital parameters of the patient, which is advantageous.

Effects and features of the second and third aspects are largely analogous to those described above in connection with the first aspect. Embodiments mentioned in relation to the first aspect are largely compatible with the second and third aspects. It is further noted that the inventive concepts relate to all possible combinations of features unless explicitly stated otherwise. Further, in a fourth aspect, there is provided a computer program product comprising a computer-readable storage medium with instructions adapted to carry out at least parts of the method as disclosed herein, and/or with instructions adapted to carry out an action in a monitoring device as disclosed herein and/or in a system as disclosed herein, when executed by a device having processing capability.

A further scope of applicability of the present disclosure will become apparent from the detailed description given below. However, it should be understood that the detailed description and specific examples, while indicating preferred embodiments of the disclosure, are given by way of illustration only, since various changes and modifications within the scope of the disclosure will become apparent to those skilled in the art from this detailed description.

Hence, it is to be understood that this disclosure is not limited to the particular component parts of the device described or steps of the methods described as such device and method may vary. It is also to be understood that the terminology used herein is for purpose of describing particular embodiments only, and is not intended to be limiting. It must be noted that, as used in the specification and the appended claim, the articles “a”, “an”, “the”, and “said” are intended to mean that there are one or more of the elements unless the context clearly dictates otherwise. Thus, for example, reference to “a unit” or “the unit” may include several devices, and the like. Furthermore, the words “comprising”, “including”, “containing” and similar wordings does not exclude other elements or steps.

### BRIEF DESCRIPTION OF THE DRAWINGS

The disclosure will by way of example be described in more detail with reference to the appended schematic drawings, which show presently preferred embodiments of the disclosure.

FIG. 1 shows a schematic overview of a system according to an embodiment of the present disclosure.

FIG. 2 shows a schematic view of a monitoring device according to an embodiment of the present disclosure.

FIG. 3 shows a block diagram of internal components comprised in the monitoring device according to an embodiment of the present disclosure.

FIG. 4 shows a schematic flowchart of a system according to an embodiment of the present disclosure.

FIG. 5 shows a schematic overview of a monitoring device according to an embodiment of the present disclosure.

FIG. 6 shows a block diagram of internal components comprised in the monitoring device according to an embodiment of the present disclosure.

FIG. 7 shows a flowchart of a method according to an aspect of the present disclosure.

### DESCRIPTION OF EMBODIMENTS

The present disclosure will now be described more fully hereinafter with reference to the accompanying drawings, in which currently preferred embodiments of the disclosure are shown. This disclosure may, however, be embodied in many different forms and should not be construed as limited to the embodiments set forth herein; rather, these embodiments are provided for thoroughness and completeness, and to fully convey the scope of the disclosure to the skilled person.

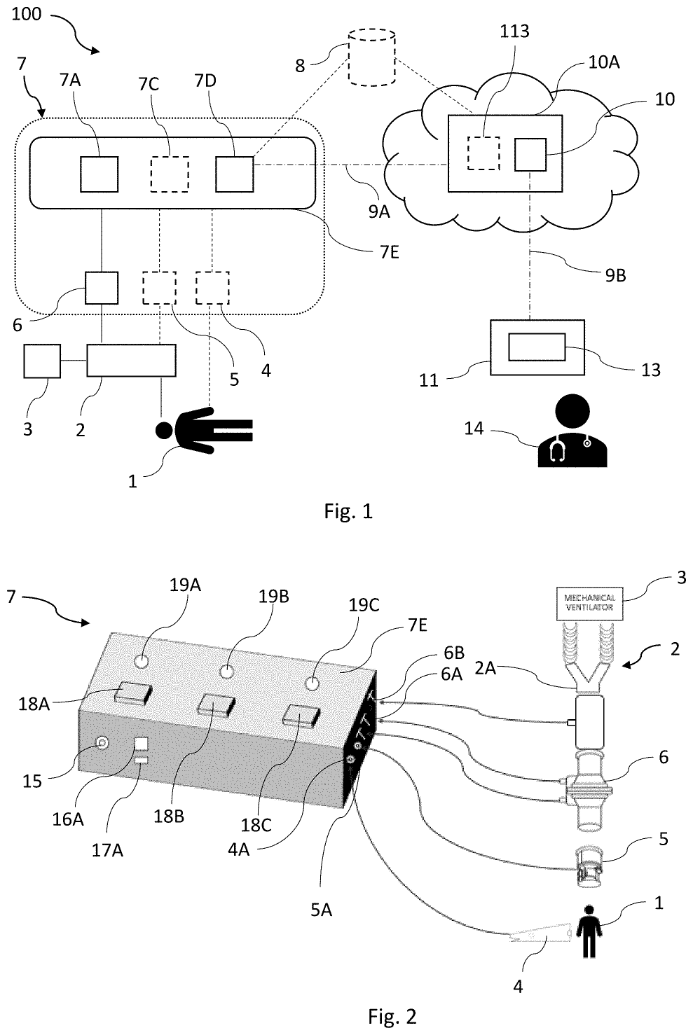

FIG. 1 shows an overview of a system 100 for remote monitoring of vital parameters of a mechanically ventilated patient 1, the vital parameters comprising at least one breathing parameter and optionally an oxygenation parameter. The system 100 comprises a monitoring device 7 which is removably arrangeable at a portion of a ventilator breathing circuit 2 provided between and in fluid connection with a mechanical ventilator 3 and an airway of the patient 1. The monitoring device 7 comprises a first sensor 6 arrangeable at the fluid connection 2 for measuring parameters related to an airflow and an airway pressure in the fluid connection 2 to obtain measurement data. Thus, the first sensor 6 is connected in-line to the ventilator breathing circuit 2 provided between the mechanical ventilator 3 and the patient 1. Preferably, the first sensor 6 is arranged in fluid connection at the patient side of a y-piece of the ventilator breathing circuit 2. Alternatively, the first sensor 6 may be arranged on a separate adaptor removably arranged between and in fluid connection with the y-piece of the ventilator breathing circuit 2 and the endotracheal tube of the patient 1. Arranging the first sensor 6 at any portion of the tubing of the ventilator breathing circuit 2 between the ventilator 3 and the patient 1 is, however, also possible within the concept of the present disclosure.

The monitoring device 7 further comprises a processor 7A adapted to receive the measurement data from the first sensor 6. The processor 7A is configured to process the measurement data into at least one breathing parameter. In some examples, the processor is configured to process the measurement data into a plurality of breathing parameters.

The monitoring device 7 also comprises a transmitter 7D adapted to transmit data comprising the breathing parameter(s) to an external device 10A. According to this embodiment, the processor 7A and the transmitter 7D are arranged in a housing 7E. The housing 7E is provided with first connection ports (described in more detail with reference to FIG. 2) for the first sensor 6, through which the first sensor is connectable to the processor 7A. The first sensor 6 is thus releasably connected to the processor 7A through the first connection ports of the housing 7E.

Further, with reference to the embodiment shown in FIG. 1, a second sensor 5, a pulse oximeter 4, and an encryptor 7C, all drawn in dashed lines, are each individually optionally comprised in the monitoring device 7. According to an embodiment, the monitoring device 7 comprises a second sensor 5, also arrangeable at the fluid connection 2 for measuring parameters related to an airflow in the fluid connection 2 between the mechanical ventilator 3 and the patient 1 to obtain second measurement data. The second sensor 5 is thus connected in-line to the ventilator breathing circuit 2 between the mechanical ventilator 3 and the patient 1. The second measurement data is transmitted to the processor 7A which is adapted to receive and process the second measurement data into a second breathing parameter or a second set of breathing parameters, which is/are transmitted to the transmitter 7D. The transmitter is in this example adapted to transmit data comprising the first and the second breathing parameter(s) to the external device 10A. Further, in this example, the housing 7E comprising the processor 7A and the transmitter 7D is provided with a second connection port (not shown) for the second sensor 5, through which the second sensor 5 is releasably connected to the processor 7A.

According to an embodiment, the monitoring device 7 further comprises a pulse oximeter 4 for measuring parameters related to the pulse and/or oxygenation of the patient to obtain fourth measurement data. The pulse oximeter 4 is arrangeable at a body of the patient 1, typically a fingertip of the patient 1. In this embodiment, the processor 7A is further adapted to receive the fourth measurement data from the pulse oximeter 4 and process this into an oxygenation parameter or a plurality of oxygenation parameters. Further, the transmitter 7D is adapted to transmit data further comprising the oxygenation parameter(s) to the external device 10. The housing 7E comprising the processor 7A and the transmitter 7D is in this example further provided with a third connection port (not shown) for the pulse oximeter 4, through which the pulse oximeter 4 is releasably connected to the processor 7A.

Finally, with regards to the monitoring device 7, according to an embodiment it further comprises an encryptor 7C for encrypting the breathing parameters and, optionally, the oxygenation parameters to encrypted data. The transmitter 7D is in this example adapted to transmit the encrypted data to the external device 10A. The encryptor 7C may be comprised in the processor 7A or provided separately from and communicatively connected to the processor 7A. The encryption may be performed using an encryption key, and the external device 10A may have access to a corresponding decryption key for decrypting the encrypted data.

Continuing with reference to the system 100 shown in FIG. 1, the system 100 further comprises the external device 10A, which is configured to receive data from the monitoring device 7. More particularly, the external device 10A is configured to receive data from the transmitter 7D of the monitoring device 7. The data can be transmitted from the monitoring device 7 to the external device 10A by means of a communication network 9A, indicated by the dash-dotted line. A communication network 9A may be any network that allows the transmission of data between network members, such as the monitoring device 7 and the external device 10A. A non-limiting example of a communication network 9A is the Internet. The external device 10A comprises a network server 10. The system 100 further comprises an intermediary device 11 connected to the network server 10. The intermediary device comprises a user interface 13 for displaying the vital parameters of the patient 1. An off-site intensivist or clinician 14 can thus monitor the vital parameters through the user interface 13 comprised by the intermediary device 11, e.g., the user interface 13 may be configured to display the vital parameters. The intermediary device 11 is here connected to the network server by means of a communication network 9B, as indicated by the dash-dotted line. The user interface, or data to be displayed by the user interface, may be requested or received by the intermediary device 11 from the network server 10. The user interface may be further adapted to request or receive data and display a user interface comprising data relating to a plurality of ventilated patients.

Also shown in dashed lines, representing devices individually optionally comprised by the system 100, are an external storage device 8 connected to the monitoring device 7 and/or to the external device 10A, and a second level encryptor system 113 comprised by the external device 10A. Thus, according to an embodiment, the system 100 comprises an external storage device 8 adapted to receive and store data transmitted from the transmitter 7D of the monitoring device 7. Data stored in the external storage device 8 can then be transferred to the network server 10 comprised in the external device 10A. The data may be further transmitted to and displayed in the user interface 13 as described above.

According to an embodiment of a system 100 comprising a monitoring device 7 comprising an encryptor 7C, the system 100 further comprises a second level encryptor system 113. The encryptor system 113 is comprised in the external device 10A for double encryption of the data encrypted by the encryptor 7C. The encryptor system 113 may be configured to double encrypt the encrypted data when stored in the network server 10. In those embodiments, the network server 10 may be further configured to decrypt, by the encryptor system 113, the double encryption of the data before transmitting it to the intermediary device 11, and the intermediary device 11 may be configured to decrypt the data before displaying it in the user interface 13. The encryption method used at the monitoring device, may, for example be hardware accelerated Advanced Encryption Standard (AES)-512 symmetric keys. The encryption method used when storing the data may, for example, be Amazon S3-managed encryption keys (SSE-S3). The encryption method used when encoding data to be transmitted to the intermediary device may, for example, be Transport Layer Security, or TLS.

In a further embodiment of the system 100 comprising an encryptor 7C and an external storage device 8, the external storage device 8 is adapted to receive and store the encrypted data transmitted from the transmitter 7D. The storage device 8 may further have access to a decryption key corresponding to the encryption key with which the data was encrypted.

With reference to FIG. 2, a schematic view of a monitoring device 7 for measuring and monitoring breathing parameters and, optionally, vital parameters e.g. in the form of oxygenation parameters or heart rate of a mechanically ventilated patient 1 according to an embodiment is shown. The monitoring device 7 may be used in connection with any mechanical ventilator 3 used in order to ventilate the patient 1. Mechanical ventilators typically work by pushing gas through a ventilator breathing circuit 2 connected to an airway of a patient. In the shown embodiment, the monitoring device 7 comprises a first sensor 6 which is a differential pressure flow sensor, connected to the patient side of a y-piece 2A of the ventilator breathing circuit 2. The monitoring device 7 further comprises a second sensor 5, which is a capnography sensor 5, also connected to a portion of the ventilator breathing circuit 2 located on the patient side of the y-piece 2A of the same. As mentioned previously in the present disclosure, the inclusion of the second sensor is optional and, thus, not critical for the monitoring device to measure and monitor breathing parameters, and more particularly ventilatory mechanics parameters, of the mechanically ventilated patient 1. The ventilator breathing circuit 2 and the thereto connected first sensor 6 and second sensor 5 are thus connected to the airway of the patient 1. In this exemplifying embodiment, the monitoring device further comprises a pulse oximeter 4 connected to the patient 1. More particularly, the pulse oximeter 4 is typically connected to a finger of the patient 1 subject to the monitoring by the monitoring device 7. However, as previously mentioned in the present disclosure, providing a pulse oximeter 4 is optional and, thus, not essential for the measuring and monitoring of the monitoring device 7. The provision of the pulse oximeter 4 is advantageous though, considering that it provides additional measurement data which may be processed and transmitted to an external device for remote monitoring.

The monitoring device 7 shown in FIG. 2 further comprises a housing 7E which houses the processor 7A, the transmitter 7D, and, optionally, the encryptor 7C of the monitoring device 7 (not shown in this figure). The housing 7E further comprises external user input interfaces 18A, 18B, 18C for entering commands to the processor 7A (not shown). In this exemplifying embodiment, the external user input interface 18A is a latching switch type button that corresponds to an ON/OFF signal to the processor 7A. The external user input interfaces 18B and 18C are momentary contact push buttons to perform a pairing routine to the communication network 9 of the system (see FIG. 1) and to send alarm signals to the off-site clinician 14. In alternative embodiments, the external user input interfaces 18A, 18B, 18C may be replaced by switches, touchscreens, or any other interface for data input to the device. Further, more than three external user input interfaces may be provided within the concept of the present disclosure.

The housing 7E further comprises external user feedback interfaces 19A, 19B, 19C to provide feedback of the status of the monitoring device 7. In this exemplifying embodiment, the external user feedback interfaces 19A, 19B, 19C are LED indicators used for local user feedback. In alternative embodiments, the user output interfaces 19A, 19B, 19C may be replaced by for example screens, visible alarms, or any other interface for user feedback. The skilled person would understand, in light of the present disclosure, that the number of user output interfaces may be adapted to the need of the monitoring device 7, and may thus be more than three.

The housing 7E also includes an Ethernet Jack 16A for a wired connection to the communication network 9, an external storage port 17A for the external storage device 8 (shown in FIG. 1), a DC plug 15 for power supply for the monitoring device 7. The monitoring device 7 may also be powered by batteries. The housing 7E further comprises connection ports 6A, 6B, 5A, 4A for sensor input to the monitoring device 7. More particularly, the housing 7E comprises first connection ports 6A, 6B to which the first sensor 6, here a differential pressure flow sensor, is releasably connected. In this embodiment, the first connection ports 6A, 6B are pneumatic ports. The housing 7E further comprises a second connection port 5A to which the second sensor 5, here a capnography sensor, is releasably connected. Finally, the housing 7E comprises a third connection port 4A to which the pulse oximeter 4 is releasably connected.

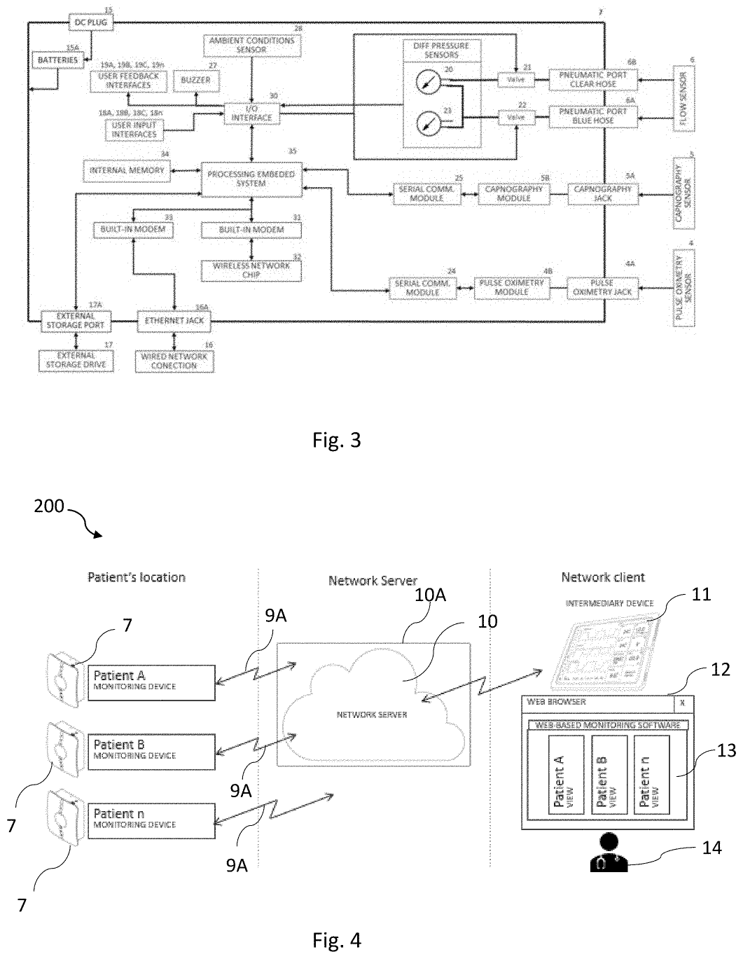

Referring to FIG. 3, a schematic block diagram illustrating the internal components of the monitoring device 7 and their interaction with external components is shown in detail. The monitoring device 7 in this example comprises a processor 7A in the form of a processing embedded system 35 and an internal memory 34. In a preferred embodiment, the internal memory 34 is a non-volatile memory such as an SD card which stores executable instructions. The processor 35 also includes a built-in read only memory (such as a ROM which stores firmware) and a random-access memory (such as a RAM for local and temporary variable storage and calculation) for controlling the operation of the monitoring device 7. It contains an I/O interface 30 which controls the opening of zeroing valves 21, 22. Zeroing valves 21, 22 are connected to differential pressure sensors 20, 23, which measure the pressure difference at two ports on the first sensor 6, here a flow sensor 6. The sensors 20, 23 further transmit such reading to I/O interface 30 which in turn transmits such signal to the processor 7A for calculating first breathing parameters comprising respiratory rate, inhale to exhale ratio, inspiratory time, expiratory time, tidal volume, peak inspiratory pressure (PIP), and positive end expiratory pressure (PEEP).

The zeroing valves 21, 22 are normally closed 3/2 way valves for allowing venting the differential pressure sensors 20, 23 to the atmosphere for zeroing calibration. Within the concept of the present disclosure, the zeroing method described can be also performed by sensors 20, 23 with auto zero features, be performed by a software, or by any other method suitable for providing zeroing calibration.

The monitoring device 7 further has two pneumatic ports 6A, 6B for respective hoses, both of which are connected to the flow sensor 6. The flow sensor 6 is a differential pressure flow sensor 6 through which a gas flow passes, creating a differential pressure between two measuring ports included in the sensor 6.

With the purpose of off-site monitoring, the processor 35 also connects with two built-in modems 31, 33. Built-in modem 33 connects to a female ethernet jack 16A so that the user has the possibility of connecting a wired network connection 16 to provide network connection to the monitoring device 7. Built-in modem 31 connects to a wireless network chip 32. The wireless chip 32 and the wired network connection 16 allow data transmission from the processor 35 to the external device 10A (not shown here). The processing embedded system 35 connects to an external storage port 17A to allow an external storage device to be connected to the monitoring device 7. In this example, the encryptor may be stored as a computer program or computer program instructions in the internal memory 34 and executed by the processing embedded system 35.

Furthermore, the I/O interface 30 connects to the external user input interfaces 18A, 18B, 18C for receiving user input, to buzzer 27 (an audible alarm), to ambient conditions sensor 28 (barometric sensor also sensing temperature and humidity) and to the user feedback interfaces 19A, 19B, 19C.

The processing embedded system 35 also connects to a serial communication module 24 which receives and transmits the signal input in serial communication protocol from a pulse oximetry module 4B, which in turn connects to the third connection port 4A. An optional external pulse oximeter 4 can be connected to the third connection port 4A for heart rate and oxygenation monitoring of the patient 1.

The processing embedded system 35 further connects to a second serial communication module 25 which receives and transmits the signal input in serial communication protocol from a second sensor module 5B which in turn is connected to the second connection port 5A. An optional external second sensor 5, here a capnography sensor, can be connected to the second connection port 5A for monitoring expired CO₂ from the patient.

Finally, serial communication protocols and modules 24 and 25 are only one of the possible methods that can be used for communicating the second sensor module 5B and the pulse oximetry module 4B with the processor 35.

Reference is now made to FIG. 4, which shows a system 200 according to an embodiment of the present disclosure for monitoring vital parameters for multiple patients. The system 200 comprises three in-situ monitoring devices 7 for monitoring vital parameters of a respective patient A, B, C, a network server 10 and network client(s) using intermediary devices 11 connected through a communication network 9. The communication network 9 is any network that allows the transmission of data between the network members, e.g. monitoring devices 7, network server 10, and intermediary devices 11. A non-limiting example of a communication network 9 is the Internet. The communication network used for the communication between the monitoring devices 7 and the network server 10, and the communication network used for the communication between the network server 10 and the intermediary devices 11 is not necessarily the same communication network. Thus, the vital parameters from the monitoring devices may be transmitted or received by the network server 10.

In this embodiment, the network server 10 is capable of communicating data from multiple monitoring devices 7 and transmit that information to more than one intermediary device 11 through a communication network 9. An intermediary device 11 may be any electronic device capable of displaying data received from the network server 10. In some examples, the intermediary devices is/are adapted for displaying a webpage, capable of navigating inside that webpage, capable of connecting to the communication network 9, and capable of receiving and/or requesting data from the network server 10. As a non-limiting example, the intermediary device 11 may be a personal computer with a web browser 12 installed and a connection to the internet. Thus, the network server may transmit the received vital parameters to the intermediary devices 11 for displaying to a clinician 14.

With further reference to FIG. 4, a clinician 14 can use the intermediary device 11 to display the user interface 13 comprising vital parameters corresponding to each of the monitored patients. The clinician 14 can visualize data from multiple monitoring devices 7 by means of the user interface 13. The user interface 13 may request or receive information from the network server 10 through the communication network 9.

An example of a setup system and procedure for the system shown in FIG. 4 will now be described. The network server 10 may be configured to receive setup data, such as a site on which the monitoring device(s) 7 will be used, user data, identifiers for the monitoring device(s) 7, and/or identifiers of the ventilators that will be monitored. The setup data may be received by the network server 10 from an API and/or a setup user interface, which may be provided by the network server 10 to an intermediary device 11. The network server 10 may further be configured to provide and receive data from a resource management user interface or API, in which monitoring schedules corresponding to a respective monitoring device 7 may be provided. The network server 10 may further be configured to receive data related to an identifier of a monitoring device 7 and associate the identifier with a site on which the monitoring device 7 is to be used. The identifier may, for example, be an identifier provided visually on the monitoring device 7, such as a QR-code. The network device 10 may be further configured to receive data related to the use of monitoring device 7, such as an identifier of the patient being monitored (for example, name, gender, age, height, weight, bed number, room number, etc.) and/or an identifier of the ventilator at which the monitoring device is arranged. In this way, the network server 10 may associate a monitoring device 7 with a specific site and/or a specific ventilator, and data relating to the patient being monitored. The data relating to the patient being monitored may be displayed by the user interface 13 together with the corresponding breathing and/or oxygenation parameters.

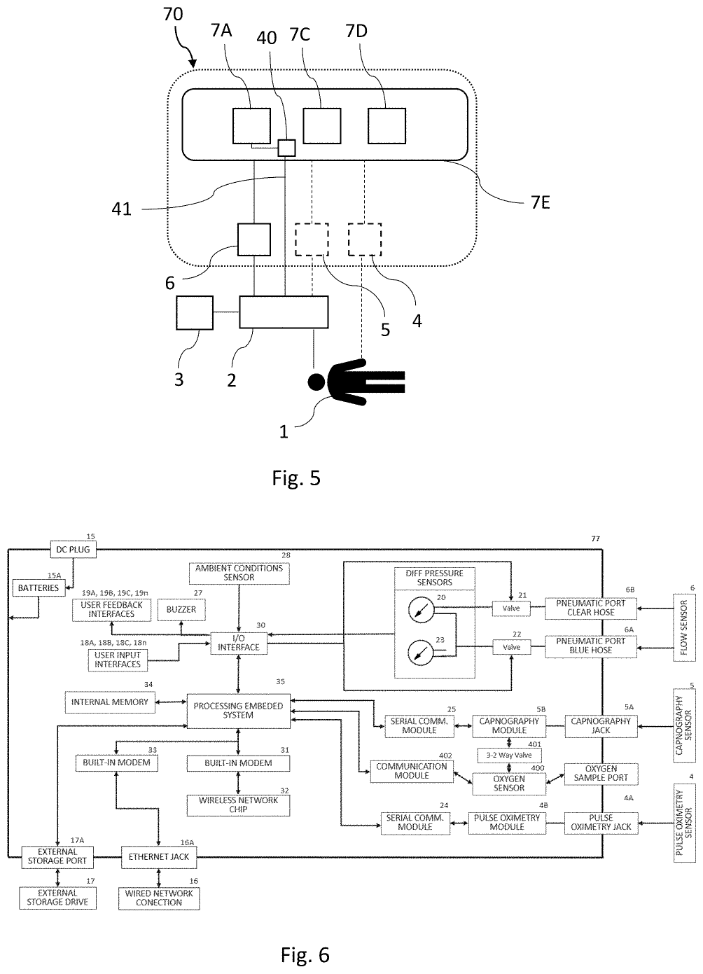

Referring to FIG. 5, a further embodiment of a monitoring device 70 according to the present disclosure is shown. The monitoring device 70 is removably arrangeable at a portion of a ventilator breathing circuit 2 provided between and in fluid connection with a mechanical ventilator 3 and an airway of a patient 1 through a first sensor 6, and optionally also through a second sensor 5, removably arranged at the ventilator breathing circuit 2, also referred to herein as the patient circuit 2. The monitoring device 70 thus comprises a first sensor 6 arrangeable at the fluid connection 2 for measuring parameters related to an airflow and an airway pressure in the fluid connection 2 to obtain measurement data, as previously described with reference to the embodiment shown in FIG. 1.

The monitoring device 70 is further similar to the embodiment described with reference to FIG. 1 in that it further comprises a processor 7A adapted to receive the measurement data from the first sensor 6. The processor 7A is configured to process the measurement data into at least one breathing parameter. In some examples, the processor is configured to process the measurement data into a plurality of breathing parameters.

The monitoring device 70 also comprises a transmitter 7D adapted to transmit data comprising the breathing parameter(s) to an external device. The processor 7A and the transmitter 7D are here arranged in a housing 7E. The monitoring device 70 also optionally comprises a second sensor 5, a pulse oximeter 4, and/or an encryptor 7C, all drawn in dashed lines and arrangeable as explained with reference to FIG. 1.

Finally, the monitoring device 70 further comprises an internal sensor 40 arranged in the housing 7E and adapted to receive a gas sample from the patient circuit 2 for measuring at least one parameter related to the gas sample to obtain measurement data. The internal sensor is here in fluid connection with the patient circuit 2 through a tubing 41, which extends between a portion of the patient circuit 2 and a port of the housing 7E which is fluidly connected with the internal sensor 40. The processor 7A is here further adapted to receive the measurement data from the internal sensor and configured to process the measurement data into at least one breathing parameter.

Referring to FIG. 6, a schematic block diagram illustrating the internal components of another embodiment of a monitoring device 77 according to the present disclosure is shown. The internal components of this embodiment correspond to those described with reference to FIG. 3. Additionally, the monitoring device 77 here comprises an internal oxygen sensor 400 connected to the capnography module 5B through directional valves 401, here 3/2 way valve. The internal oxygen sensor 400 is configured to receive a gas sample from the capnography module 5B to measure the oxygen concentration therein. The directional valves 401 may be configured to allow flow of a gas sample from the capnography module 5B to the internal oxygen sensor 400 only when there is evidence that the gas sample corresponds to a gas delivered in the inspiration phase of the mechanical ventilation of the patient. Such evidence may for example correspond to a low level of CO₂ measured by the capnography module 5B, thus indicating that the sample corresponds to fresh gas. The internal oxygen sensor 400 is further connected to a communication module 402 through which the measurement data from the oxygen sensor is transmitted to the processor 35 for processing.

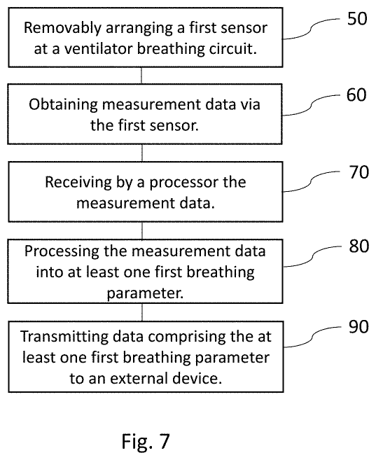

Finally, with reference to FIG. 7, a method for measuring and remote monitoring of breathing parameters and, optionally, oxygenation parameters of a mechanically ventilated patient is shown. The method comprises the steps of removably arranging 50a first sensor at a portion of a ventilator breathing circuit provided between and in fluid connection with a mechanical ventilator and the airway of the patient; obtaining measurement data (60), via the first sensor, related to an airflow and an airway pressure in the fluid connection; receiving (70), by a processor, the measurement data from the first sensor; processing (80), by the processor, the measurement data into at least one first breathing parameter; and transmitting (90), by a transmitter connected to the processor, data comprising the at least one first breathing parameter to an external device.

The person skilled in the art realizes that the present disclosure by no means is limited to the preferred embodiments described above. On the contrary, many modifications and variations are possible within the scope of the appended claims.

Additionally, variations to the disclosed embodiments can be understood and effected by the skilled person in practicing the claimed disclosure, from a study of the drawings, the disclosure, and the appended claims.

1. A monitoring device for measuring and monitoring breathing parameters and, optionally, oxygenation and/or vital sign parameters of a mechanically ventilated patient, the monitoring device being removably arrangeable at a portion of a ventilator breathing circuit provided between and in fluid connection with a mechanical ventilator and an airway of the patient, the monitoring device comprising:
a first sensor arrangeable at the fluid connection for measuring at least one parameter related to an airflow in the fluid connection to obtain measurement data;
a processor adapted to receive the measurement data from the first sensor and configured to process the measurement data into at least one breathing parameter; and
a transmitter adapted to transmit data comprising the at least one breathing parameter to an external device.

2. The monitoring device according to claim 1, wherein the first sensor is a flow sensor.

3. The monitoring device according to claim 2, wherein the flow sensor is a differential pressure flow sensor.

4. The monitoring device according to claim 1, wherein the at least one breathing parameter comprises ventilatory mechanics data.

5. The monitoring device according to claim 4, wherein the ventilator mechanics data comprises at least one of airway pressure, gas flow, respiratory rate, inhale to exhale ratio, inspiratory time, expiratory time, tidal volume, peak inspiratory pressure, positive end expiratory pressure, and fraction of inspired oxygen.

6. The monitoring device according to claim 1, further comprising a second sensor arrangeable at the fluid connection for measuring at least one parameter related to an airflow in the fluid connection to obtain second measurement data, and wherein the processor is adapted to receive the second measurement data from the second sensor and configured to process the second measurement data into at least one breathing parameter.

7. The monitoring device according to claim 6, wherein the second sensor is a capnography sensor.

8. The monitoring device according to claim 6, wherein the second sensor is an oxygen sensor.

9. The monitoring device according to claim 7, further comprising a third sensor arrangeable at the ventilator breathing circuit for measuring at least one parameter related to an airflow therein to obtain third measurement data, and wherein the processor is adapted to receive the third measurement data from the third sensor and configured to process the third measurement data into at least one breathing parameter, and wherein the third sensor is an oxygen sensor.

10. The monitoring device according to claim 1, further comprising an internal sensor which is arranged in a housing of the monitoring device and adapted to receive a gas sample from the ventilator breathing circuit for measuring at least one parameter related to the gas sample to obtain measurement data, and wherein the processor is adapted to receive the measurement data from the internal sensor and configured to process the measurement data into at least one breathing parameter.

11. The monitoring device according to claim 1, further comprising a pulse oximeter for measuring at least one parameter related to pulse and/or oxygenation of the patient to obtain fourth measurement data, and wherein the processor is adapted to receive the fourth measurement data from the pulse oximeter and process the fourth measurement data into at least one oxygenation parameter, and wherein the transmitter is adapted to transmit data comprising the at least one oxygenation parameter.

12. The monitoring device according to claim 1, wherein the portion of the ventilator breathing circuit at which the measuring device is removably arrangeable is the patient end of a y-piece of the ventilator breathing circuit, the inspiration limb of the ventilator breathing circuit, and/or the expiration limb of the ventilator breathing circuit.

13. The monitoring device according to claim 1, wherein any of the first sensor, the second sensor, the third sensor, and the pulse oximeter is releasably connected to the processor.

14. The monitoring device according to claim 1, wherein the processor comprises an encryptor for encrypting the at least one breathing parameter and/or the at least one oxygenation parameter to encrypted data, and wherein the transmitter is adapted to transmit the encrypted data to the external device.

15. A system for measuring and remote monitoring of breathing parameters and, optionally, oxygenation and/or vital sign parameters of a mechanically ventilated patient, the system comprising:
a monitoring device according to any one of the preceding claims associated with the patient,
an external device configured to receive data from the monitoring device, the external device comprising a network server, and
an intermediary device connected to the network server and comprising a user interface for displaying the breathing parameters and, optionally, oxygenation and/or vital sign parameters.

16. The system according to claim 15, further comprising an external storage device adapted to receive and store data transmitted from the transmitter of the monitoring device.

17. The system according to claim 15, comprising a plurality of monitoring devices, each associated with a respective patient, and wherein the external device is configured to receive data from the plurality of monitoring devices.

18. The system according to claim 17, wherein the user interface is configured to display vital parameters for a plurality of patients.

19. The system according to claim 15, comprising a plurality of intermediary devices connected to the network server.

20. A method for measuring and remote monitoring of breathing parameters and, optionally, oxygenation and/or vital sign parameters of a mechanically ventilated patient, the method comprising the steps of:
removably arranging a first sensor at a portion of a ventilator breathing circuit provided between and in fluid connection with a mechanical ventilator and the airway of the patient;
obtaining measurement data, via the first sensor, related to an airflow and an airway pressure in the fluid connection;
receiving, by a processor, the measurement data from the first sensor;
processing, by the processor, the measurement data into at least one first breathing parameter; and
transmitting, by a transmitter connected to the processor, data comprising the at least one first breathing parameter to an external device.

21. The method according to claim 20, further comprising
removably arranging a second sensor at the portion of the ventilator breathing circuit;
obtaining second measurement data, via the second sensor, related to the airflow in the fluid connection;
receiving, by the processor, the second measurement data from the second sensor;
processing, by the processor, the measurement data into at least one second breathing parameter; and
wherein the transmitting comprises transmitting data comprising the first and the second breathing parameters to an external device.

22. The method according to claim 20, further comprising removably arranging a fourth sensor at a body of the patient;
obtaining fourth measurement data, via the fourth sensor, related to a pulse and/or an oxygenation and/or a vital sign of the patient;
receiving, by the processor, the fourth measurement data from the fourth sensor;
processing, by the processor, the measurement data into at least one oxygenation or vital sign parameter; and
wherein the transmitting comprises transmitting data comprising the first and the second breathing parameters, and the at least one oxygenation and/or vital sign parameter to an external device.

23. The method according to claim 20, further comprising encrypting the at least one first breathing parameter, the at least one second breathing parameter, the at least one oxygenation parameter, and/or the data before the transmitting to the external device.

24. The method according to claim 23, further comprising decrypting, by the external device, data received from the transmitter.

25. The method according to claim 20, wherein the first sensor is a differential pressure flow sensor.

26. The method according to claim 21, wherein the second sensor is one of a capnography sensor and an oxygen sensor.

27. The method according to claim 22, wherein the fourth sensor is a pulse oximeter.

28. The method according to claim 20, further comprising receiving, by an intermediary device connected to the external device, data related to a user interface for displaying the breathing parameters and, optionally, the oxygenation and/or vital sign parameters; and displaying, by the intermediary device, the user interface.

29. The method according to claim 28, wherein the user interface is configured to display vital parameters for a plurality of patients.

30. A computer program product comprising a computer-readable storage medium with instructions adapted to carry out the method of claim 20 when executed by a device having processing capability.
